(19) United States
(12) Patent Application Publication
(10) Pub. No.: US 2025/0191673 A1
(43) Pub. Date: Jun. 12, 2025

(54) Scoring of Whole Protein MSMS Spectra Based on a Bond Relevance Score

(71) Applicant: DH Technologies Development Pte. Ltd., Singapore (SG)

(72) Inventors: Stephen A. TATE, Barrie (CA); Claudia ALVAREZ, Vaughan (CA); Lyle Lorrence BURTON, Woodbridge (CA)

(21) Appl. No.: 18/856,396

(22) PCT Filed: Mar. 23, 2023
(86) PCT No.: PCT/IB2023/052881

§ 371 Date: Oct. 11, 2024

Related U.S. Application Data
(60) Provisional application No. 63/362,883, filed on Apr. 12, 2022.

(51) Int. Cl. G16B 15/20; G16B 40/10; G16C 20/20

(52) U.S. Cl. CPC G16B 15/20; G16B 40/10; G16C 20/20

(57) ABSTRACT

A method for scoring a bond of a polymeric compound of a sample from evidence determined from an experimental a product ion spectrum measured from the sample. At least one experimental product ion spectrum is received for the polymeric compound. One or more product ions of the at least one spectrum are assigned to at least one bond of the polymeric compound. At least two different types bond level scores are calculated for the at least one bond from the assigned matching one or more product ions. The at least two different bond level scores are combined, producing a combined bond score for the at least one bond. Additionally, a combined bond score is found for each bond of the polymeric compound and the combined bond scores are calculated as a function of the position of the bonds in the polymeric compound, producing a score profile for the polymeric compound.

### RELATED APPLICATIONS

This application claims the benefit of U.S. Provisional Patent Application Ser. No. 63/362,883, filed on Apr. 12, 2022, the content of which is incorporated by reference herein in its entirety.

### FIELD

The teachings herein relate to scoring a bond of a polymeric compound from a product ion spectrum.

### INTRODUCTION

### Automatic Correlation of Product Ion and Charge State Information

Tandem mass spectrometry (or mass spectrometry/mass spectrometry MS/MS) spectra generated from a top or middle-down fragmentation of a protein or large peptide (>5 Kda) generates a significant number of fragments that cover the complete sequence. Typically, each fragment is assigned a specific product ion, either a c′-(N-terminal) or z′ (C-terminal) type product ion, by a match of mass and isotope distribution.

Currently, however, there is no automatic correlation among the fragments, nor is there any correlation among the representations of the different fragments at multiple charge states or other factors. Instead, users manually evaluate different peak properties through the use of expert knowledge to determine if the match is correct and whether the fragment can be assigned to a specific spectral peak or charge cluster. An expert user also manually correlates the identification through the use of complementary c′ and z′ ions.

As a result, additional systems and methods are needed to automatically correlate the information provided from different product ions and different charge states found when a bond of a protein or other polymeric compound is broken.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Fragmentation Techniques Background

Electron-based dissociation (ExD), ultraviolet photodissociation (UVPD), infrared photodissociation (IRMPD), and collision-induced dissociation (CID) are often used as fragmentation techniques for tandem mass spectrometry (MS/MS). CID is the most conventional technique for dissociation in tandem mass spectrometers.

ExD can include, but is not limited to, electron-induced dissociation (EID), electron impact excitation in organics (EIEIO), electron capture dissociation (ECD), or electron transfer dissociation (ETD).

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### SUMMARY

The teachings herein relate to scoring a bond of a polymeric compound from a product ion spectrum. More particularly the teachings herein relate to systems and methods for calculating at least two bond level scores from a product ion spectrum of a polymeric compound for a bond of the polymeric compound and combining those scores into a combined bond score for the bond.

The systems and methods herein can be performed in conjunction with a processor, controller, or computer system, such as the computer system of FIG. 1.

A system, method, and computer program product are disclosed for scoring a bond of a polymeric compound from a product ion spectrum. A sequence and at least one product ion spectrum are received for a polymeric compound. One or more theoretical product ions resulting from the cleavage of at least one bond of the sequence are calculated. The one or more theoretical product ions are compared to the at least one spectrum. One or more matching product ions of the at least one spectrum are produced that are assigned to the at least one bond.

At least two different types of bond level scores are calculated for the at least one bond from the assigned matching one or more product ions. The at least two different types of bond level scores are combined. A combined bond score is produced for the at least one bond.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary flowchart showing a method for combining scores for a sequence match, in accordance with various embodiments.

FIG. 3 is an exemplary plot of combined or total bond level scores calculated from an experimental product ion spectrum for a sequence that are plotted as a function of bond number or position, in accordance with various embodiments.

FIG. 4 is an exemplary plot of combined or total bond level scores calculated from a different experimental product ion spectrum than the one used in FIG. 3 for the same sequence used in FIG. 3, in accordance with various embodiments.

FIG. 5 is a schematic diagram of a system for scoring a bond of a polymeric compound from a product ion spectrum, in accordance with various embodiments.

FIG. 6 is an exemplary flowchart showing a method for scoring a bond of a polymeric compound from a product ion spectrum, in accordance with various embodiments.

FIG. 7 is a schematic diagram of a system that includes one or more distinct software modules and that performs a method for scoring a bond of a polymeric compound from a product ion spectrum, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

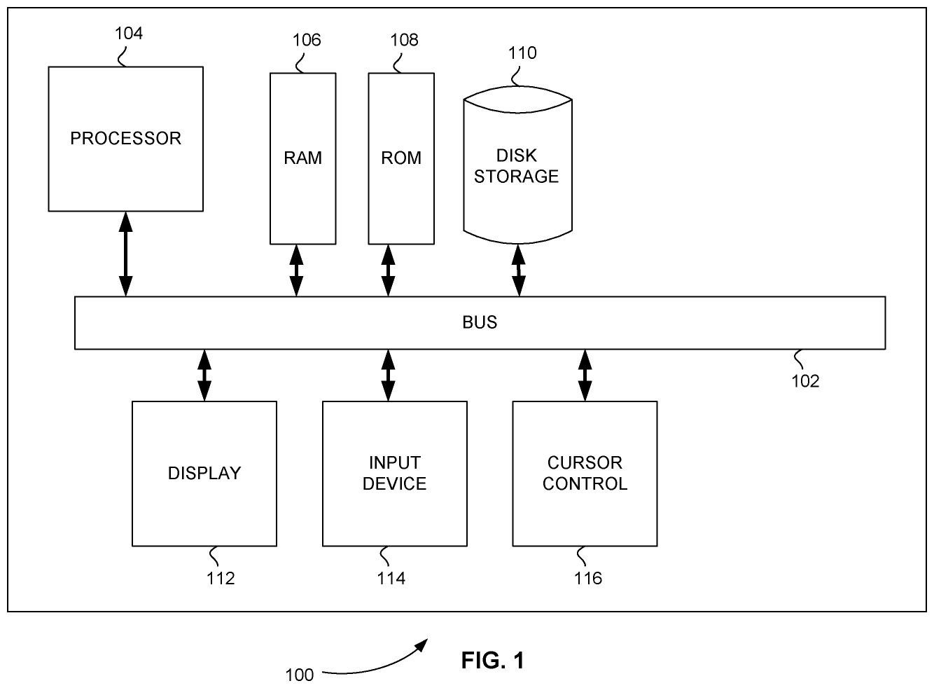

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein.

Alternatively, hard-wired circuitry may be used in place of or in combination with software instructions to implement the present teachings. For example, the present teachings may also be implemented with programmable artificial intelligence (AI) chips with only the encoder neural network programmed—to allow for performance and decreased cost. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media and volatile media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Combining Different Bond Level Scores

As described above, product ion spectra generated from a top or middle-down fragmentation of a polymeric compound include a significant number of fragments. Typically, each fragment is assigned a specific product ion, by a match of mass and isotope distribution.

Currently, however, there is no automatic correlation among the fragments, nor is there any correlation among the representations of the different fragments at multiple charge states or other factors.

As a result, additional systems and methods are needed to automatically correlate the information provided from different product ions and different charge states found when a bond of a protein or other polymeric compound is broken.

In various embodiments, a method for scoring a specific bond through the evidence that the bond has been broken is provided. This method provides a mechanism for rapid review of the sequence match and also the quality of the data.

An end-user desires information on the evidence for a polymeric compound found being of the correct sequence. The sequence is defined through the presence of a bond between two different residues and the relevant order of the residues. During electron-capture dissociation (ECD) fragmentation of a polymeric chain, for example, evidence of the bond is provided by the presence of the complementary fragment ions being present (complementary score), with the fragment resulting from the N-terminal chain or the C-terminal chain. Each of these fragments can also be represented in the data through the presence of multiple charge elements (charge score), the quality of the spectral metrics, such as ppm error (error score), and also the quality of spectral pattern match (pattern score).

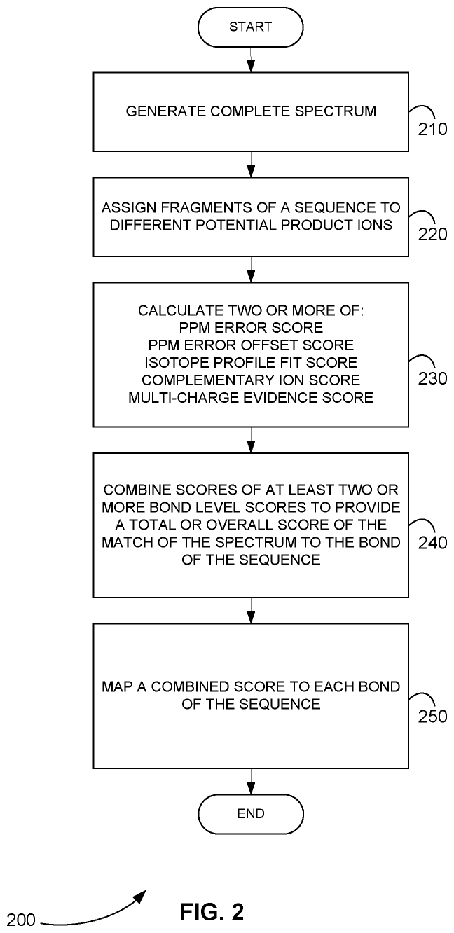

FIG. 2 is an exemplary flowchart showing a method 200 for combining scores for a sequence match, in accordance with various embodiments.

In step 210 of method 200, a complete product ion spectrum is generated for a polymeric compound.

In step 220, fragments of a sequence are assigned to different product ions of the product ion spectrum.

In step 230, two or more bond level scores are calculated for the assigned different product ions. These bond level scores can include, but are not limited to, a parts per million (ppm) error or mass error score, a ppm or mass error offset score, an isotope profile fit score, a complementary ion score, and a multi-charge evidence score. The mass error score reflects the difference between the theoretical mass-to-charge ratio (m/z) value of the theoretical product ion and the experimentally measured m/z value of the measured product ion. The mass error offset score reflects an average deviation in the mass error of multiple product ions. An isotope profile fit score or isotope pattern match score reflects an intensity profile match of experimental versus theoretical profiles. A complementary ion score or complement fragment match score reflects the identification of a complementary ion, such as a z-ion for a c-ion or a b-ion for a y-ion. A multi-charge evidence score or multiple charge state multiplier score reflects the presence of multiple charge states of matching experimental ions.

In various embodiments, additional bond level scores can be used. A mass error trend with m/z score, for example, reflects the linearity of the trend in mass error with increasing m/z value. The average or weighted average for the isotope m/z error score, for example, reflects the average or weighted average error of the isotope m/z values. The predicted charge state correlation score, for example, reflects the prediction of charge by residues to the likely visible charges. The isotope cluster signal-to-noise score, for example, reflects the signal-to-noise of the isotope cluster. The fit or purity to the measured spectra scores, for example, compares in silico and measured spectra using fit and purity scores.

In step 240, scores of at least two or more bond level scores are combined to provide a total or overall score of the match of the spectrum to the bond of the sequence.

In step 250, a combined score is mapped to each bond of the sequence.

Many of the scores described above have been calculated and used conventionally. However, these scores have not been combined to provide a combined or total bond level score. In various embodiments, an appropriate method for combining scores is used, such as summing scores, calculating a mean, median, or mode, or calculating a nonlinear combination of scores.

In various embodiments, a combined or total bond level score is calculated for each bond of the sequence. These combined or total bond level scores can then be stored as a function of bond number or position, providing a total bond score profile for the sequence. Profiles of such scores using some trend vs the bond position can be envisioned, either in a 2D or 3D representation.

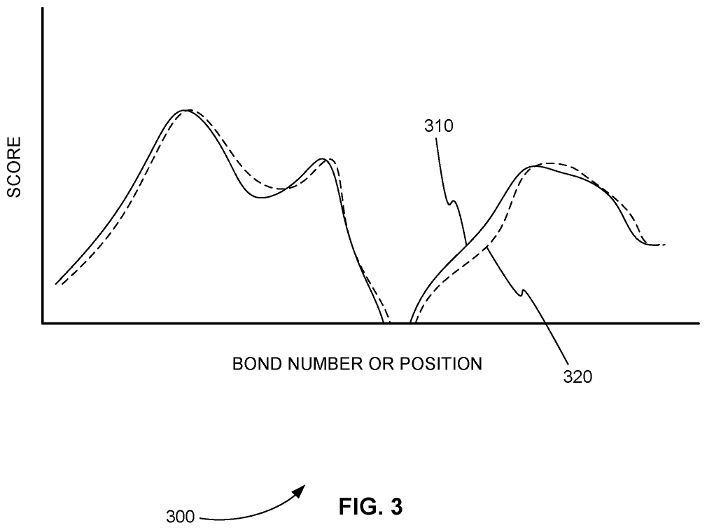

FIG. 3 is an exemplary plot 300 of combined or total bond level scores calculated from an experimental product ion spectrum for a sequence that are plotted as a function of bond number or position, in accordance with various embodiments. If a standard is adopted for calculating total bond level scores, standard profiles can be stored for known sequences. For example, in FIG. 3, standard profile 320 depicts the total bond level scores of a standard profile that is known for the same sequence for which experimental profile 310 is calculated.

Determining a match for the sequence is then simply a matter of comparing experimental profile 310 and standard profile 320. As shown in FIG. 3, a comparison of these profiles shows that the experimental product ion spectrum includes the sequence. Experimental profile 310 and standard profile 320 are similar.

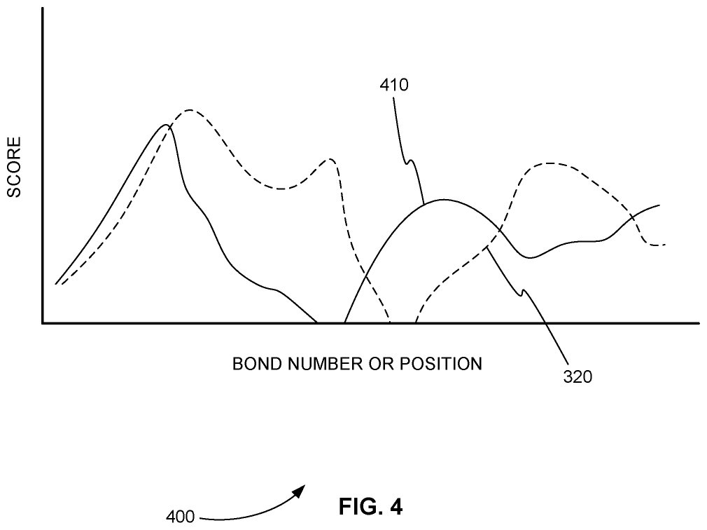

FIG. 4 is an exemplary plot 400 of combined or total bond level scores calculated from a different experimental product ion spectrum than the one used in FIG. 3 for the same sequence used in FIG. 3, in accordance with various embodiments. In FIG. 4, experimental profile 410 is calculated from a different experimental product ion spectrum than the one used in FIG. 3. Standard profile 320 depicts the total bond level scores of the same standard profile used in FIG. 3. As shown in FIG. 3, a comparison of these profiles quickly shows that the experimental product ion spectrum now used does not include the sequence. Experimental profile 410 and standard profile 320 are not similar.

FIGS. 3 and 4 show that creating a total bond level score for bonds of sequence can provide a more direct method for an end-user to review the data of the grouped fragments. Aggregating the bond scores into a bond score single profile makes it possible to identify a sequence from a simple comparison of profiles.

### System for Scoring a Bond of a Polymeric Compound

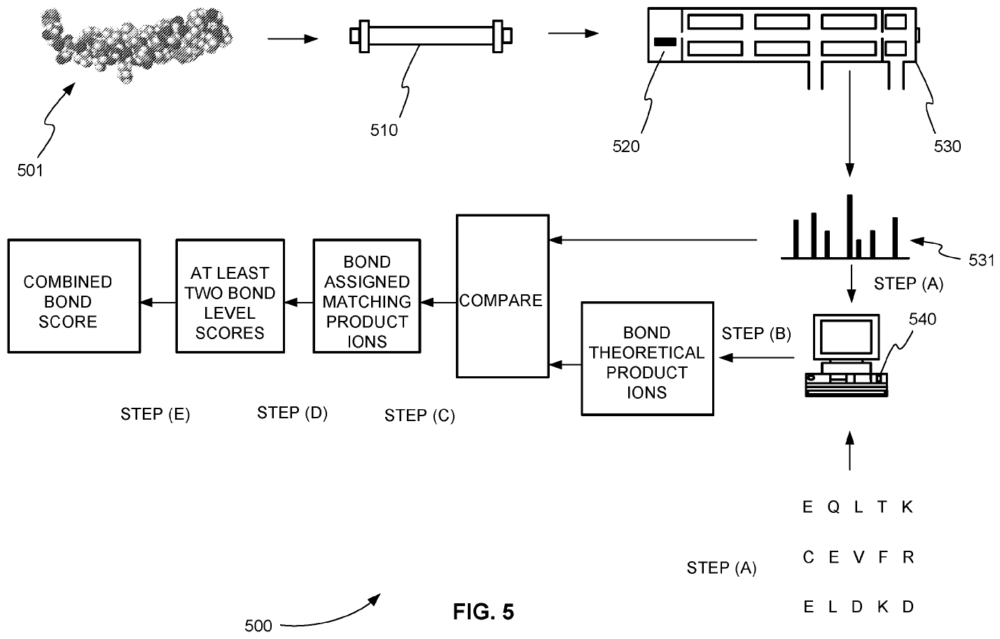

FIG. 5 is a schematic diagram 500 of a system for scoring a bond of a polymeric compound from a product ion spectrum, in accordance with various embodiments. The system includes processor 540. Processor 540 can be, but is not limited to, a controller, a computer, a microprocessor, the computer system of FIG. 1, or any device capable of analyzing data. Processor 540 can also be any device capable of sending and receiving control signals and data.

In a step (A), processor 540 receives a sequence and at least one product ion spectrum 531 for a polymeric compound. In step (B), processor 540 calculates one or more theoretical product ions resulting from the cleavage of at least one bond of the sequence. In step (C), processor 540 compares the one or more theoretical product ions to spectrum 531. One or more matching product ions of spectrum 531 are assigned to the at least one bond. In step (D), processor 540 calculates at least two different types of bond level scores for the at least one bond from the assigned matching one or more product ions. In step (E), processor 540 combines the at least two different types of bond level scores. A combined bond score is produced for the at least one bond.

In various embodiments, spectrum 531 is produced using ECD.

In various embodiments, at least one of the at least two different types of bond level scores includes a charge score that indicates a number of different charge states found for the assigned matching one or more product ions.

In various embodiments, at least one of the at least two different types of bond level scores includes a mass score that indicates how well m/z values found for the assigned matching one or more product ions match expected m/z values of matching theoretical product ions.

In various embodiments, at least one of the at least two different types of bond level scores includes a mass offset score that indicates how well an average mass error found for the assigned matching one or more product ions matches an expected mass error.

In various embodiments, at least one of the at least two different types of bond level scores includes an isotope pattern score that indicates how well an isotope pattern found for the assigned matching one or more product ions matches an expected isotope pattern of matching theoretical product ions.

In various embodiments, processor 540 combines the at least two different types of bond level scores using a summation.

In various embodiments, processor 540 combines the at least two different types of bond level scores using an average.

In various embodiments, processor 540 combines the at least two different types of bond level scores using a median.

In various embodiments, processor 540 combines the at least two different types of bond level scores using a nonlinear combination method.

In various embodiments, processor 540 performs steps (B)-(E) for each bond of the polymeric compound. A plurality of combined bond scores are produced for the polymeric compound.

In various embodiments, processor 540 further calculates the plurality of combined bond scores as a function of the position of the corresponding bonds in the polymeric compound. A score profile is produced for the polymeric compound.

In various embodiments, processor 540 further displays a plot of score versus bond position of the score profile for the polymeric compound on a display device.

In various embodiments, the system of FIG. 5 further includes mass spectrometer 530 that measures mass spectrum 531 and sends mass spectrum 531 to processor 540. Ion source device 520 of mass spectrometer 530 ionizes separated fragments of compound 501 or only compound 501, producing an ion beam. Ion source device 520 is controlled by processor 540, for example. Ion source device 520 is shown as a component of mass spectrometer 530. In various alternative embodiments, ion source device 520 is a separate device. Ion source device 520 can be, but is not limited to, an electrospray ion source (ESI) device or a chemical ionization (CI) source device such as an atmospheric pressure chemical ionization source (APCI) device or an atmospheric pressure photoionization (APPI) source device.

Mass spectrometer 530 mass analyzes product ions of compound 501 or selects and fragments compound 501 and mass analyzes product ions of compound 501 from the ion beam at a plurality of different times. Mass spectrum 531 is produced for compound 501. Mass spectrometer 530 is controlled by processor 540, for example.

In the system of FIG. 5, mass spectrometer 530 is shown as a triple quadrupole device. One of ordinary skill in the art can appreciate that any component of mass spectrometer 530 can include other types of mass spectrometry devices including, but not limited to, ion traps, orbitraps, time-of-flight (TOF) devices, ion mobility devices, or Fourier transform ion cyclotron resonance (FT-ICR) devices.

In various embodiments, the system of FIG. 5 further includes additional device 510 that affects compound 501, providing the at least one additional dimension. As shown in FIG. 5, additional device 510 is an LC device and the at least one additional dimension or spectral data provided is retention time. In various alternative embodiments, additional device 510 can be, but is not limited to, a gas chromatography (GC) device, capillary electrophoresis (CE) device, an ion mobility spectrometry (IMS) device, or a differential mobility spectrometry (DMS) device. In still further embodiments, additional device 510 is not used and the at least one additional dimension or spectral data provided is precursor ion m/z and is provided by mass spectrometer 530 operating in a precursor ion scanning mode.

### Method for Scoring a Bond of a Polymeric Compound

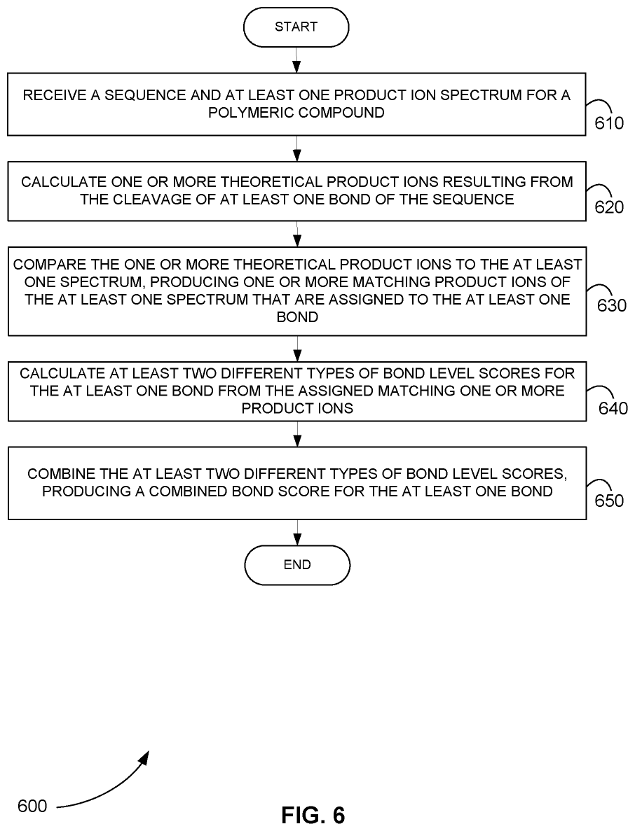

FIG. 6 is an exemplary flowchart showing a method 600 for scoring a bond of a polymeric compound from a product ion spectrum, in accordance with various embodiments.

In step 610 of method 600, a sequence and at least one product ion spectrum are received for a polymeric compound.

In step 620, one or more theoretical product ions resulting from the cleavage of at least one bond of the sequence are calculated.

In step 630, the one or more theoretical product ions are compared to the at least one spectrum. One or more matching product ions of the at least one spectrum are produced that are assigned to the at least one bond.

In step 640, at least two different types of bond level scores are calculated for the at least one bond from the assigned matching one or more product ions.

In step 650, the at least two different types of bond level scores are combined. A combined bond score is produced for the at least one bond.

### Computer Program Product for Scoring a Bond of a Polymeric Compound

In various embodiments, a computer program product includes a non-transitory tangible computer-readable storage medium whose contents include a program with instructions being executed on a processor so as to perform a method for scoring a bond of a polymeric compound from a product ion spectrum. This method is performed by a system that includes one or more distinct software modules.

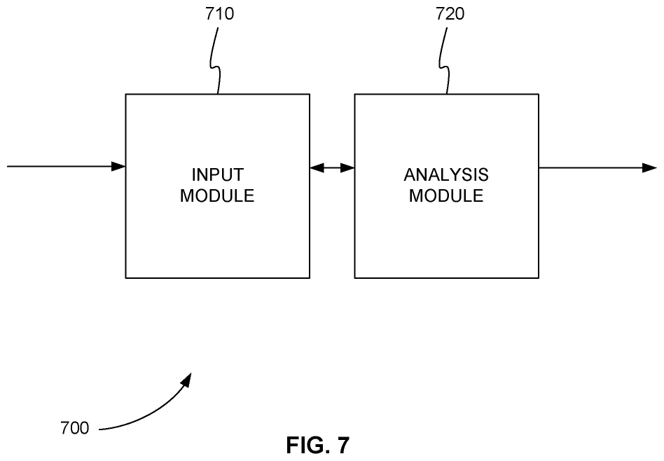

FIG. 7 is a schematic diagram of a system 700 that includes one or more distinct software modules and that performs a method for scoring a bond of a polymeric compound from a product ion spectrum, in accordance with various embodiments. System 700 includes input module 710 and analysis module 720.

Input module 710 receives a sequence and at least one product ion spectrum for a polymeric compound.

Analysis module 720 calculates one or more theoretical product ions resulting from the cleavage of at least one bond of the sequence. Analysis module 720 compares the one or more theoretical product ions to the at least one spectrum. One or more matching product ions of the at least one spectrum are produced that are assigned to the at least one bond.

Analysis module 720 calculates at least two different types of bond level scores for at least one bond from the assigned matching one or more product ions. Analysis module 720 combines the at least two different types of bond level scores. A combined bond score is produced for the at least one bond.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A method for scoring a bond of a polymeric compound from a product ion spectrum, comprising:
(a) receiving a sequence and at least one product ion spectrum for a polymeric compound;
(b) calculating one or more theoretical product ions resulting from the cleavage of at least one bond of the sequence;
(c) comparing the one or more theoretical product ions to the at least one spectrum, producing one or more matching product ions of the at least one spectrum that are assigned to the at least one bond;
(d) calculating at least two different types of bond level scores for the at least one bond from the assigned matching one or more product ions; and
(e) combining the at least two different types of bond level scores, producing a combined bond score for the at least one bond.

2. The method of claim 1, wherein the at least one spectrum is produced using electron capture dissociation (ECD).

3. The method of claim 1, wherein at least one of the at least two different types of bond level scores comprises a charge score that indicates a number of different charge states found for the assigned matching one or more product ions.

4. The method of claim 1, wherein at least one of the at least two different types of bond level scores comprises a mass score that indicates how well mass-to-charge ratio (m/z) values found for the assigned matching one or more product ions match expected m/z values of matching theoretical product ions.

5. The method of claim 1, wherein at least one of the at least two different types of bond level scores comprises a mass offset score that indicates how well an average mass error found for the assigned matching one or more product ions matches an expected mass error.

6. The method of claim 1, wherein at least one of the at least two different types of bond level scores comprises an isotope pattern score that indicates how well an isotope pattern found for the assigned matching one or more product ions matches an expected isotope pattern of matching theoretical product ions.

7. The method of claim 1, wherein combining the at least two different types of bond level scores comprises using a summation.

8. The method of claim 1, wherein combining the at least two different types of bond level scores comprises using an average.

9. The method of claim 1, wherein combining the at least two different types of bond level scores comprises using a median.

10. The method of claim 1, wherein combining the at least two different types of bond level scores comprises using a nonlinear combination method.

11. The method of claim 1, further comprising performing steps (b)-(e) for each bond of the polymeric compound, producing a plurality of combined bond scores for the polymeric compound.

12. The method of claim 11, further comprising calculating the plurality of combined bond scores as a function of a position of the corresponding bonds in the polymeric compound, producing a score profile for the polymeric compound.

13. The method of claim 12, further comprising displaying a plot of score versus bond position of the score profile for the polymeric compound on a display device.

14. A computer program product, comprising a non-transitory tangible computer-readable storage medium whose contents cause a processor to perform a method for scoring a bond of a polymeric compound from a product ion spectrum, comprising:
providing a system, wherein the system comprises one or more distinct software modules, and wherein the distinct software modules comprise an input module and an analysis module;
receiving a sequence and at least one product ion spectrum for a polymeric compound using the input module;
calculating one or more theoretical product ions resulting from the cleavage of at least one bond of the sequence using the analysis module;
comparing the one or more theoretical product ions to the at least one spectrum using the analysis module, producing one or more matching product ions of the at least one spectrum that are assigned to the at least one bond;
calculating at least two different types of bond level scores for at least one bond from the assigned matching one or more product ions using the analysis module; and
combining the at least two different types of bond level scores producing a combined bond score for the at least one bond using the analysis module.

15. A system for scoring a bond of a polymeric compound from a product ion spectrum, comprising:
a processor that
receives a sequence and at least one product ion spectrum for a polymeric compound,
calculates one or more theoretical product ions resulting from the cleavage of at least one bond of the sequence,
compares the one or more theoretical product ions to the at least one spectrum, producing one or more matching product ions of the at least one spectrum that are assigned to the at least one bond,
calculates at least two different types of bond level scores for at least one bond from the assigned matching one or more product ions, and
combines the at least two different types of bond level scores producing a combined bond score for the at least one bond.
